(19) United States
(12) Patent Application Publication
(10) Pub. No.: US 2003/0158460 A1
(43) Pub. Date: Aug. 21, 2003

(54) Radiolabeled solid surfaces and process for making them

(76) Inventors: Chaim Sukenik, D.N. Shimshon (IL); Harold Jacob, Cedarhurst, NY (US); Marc Sheldon Berridge, Solon, OH (US)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/043,491

(22) Filed: Jan. 11, 2002

(30) Foreign Application Priority Data
Jan. 11, 2001 (IL) 140874

(51) Int. Cl. A61N 5/00; A61F 2/06; A61L 2/00

(52) U.S. Cl. 600/1; 623/1.44; 427/2.24

(57) ABSTRACT

A device, which comprises a surface layer that has incorporated therein at least one radioactive nuclide. The surface layer is a self-assembled layer, particularly an anchored SAM that is selected from the group consisting of monolayers or films anchored by siloxane, thiol, amine or phosphonate. The surface layer may be formed of a radioactive material that has been activated to induce radioactivity therein after its final formation. The device may comprise a chemically functionalized SAM incorporating radionuclides attached at the surface of the device. The device may be a temporary or permanent therapeutic implant, such as a stent.

### FIELD OF THE INVENTION

The invention relates to devices containing radioactive nuclides, such as for instance, but not exclusively, electronic devices and biomedical implants, particularly devices containing radiolabeled solid surfaces. The invention also relates to a process for creating radiolabeled solid surfaces by in situ modification of self-assembled coatings, particularly monolayer films.

### BACKGROUND OF THE INVENTION

Various devices incorporating radioactive nuclides are known in the art for various purposes. For instance, therapeutic devices are known which contain or are made of a material incorporating radioactive nuclides. However, it is believed that such devices are not completely satisfactory, both as to their preparation and their use. The art has developed the ability to control surface properties and compositions having little or no effect on the bulk of a material. Such control is emerging as an increasingly important strategy in the design of complex devices in various fields. One way of controlling surface properties of devices is based on the use of SAMs (Self-Assembled Organic Monolayers).

The creation of siloxane-anchored SAMs involves the attachment of a self-assembled organic thin film onto an oxide layer on silicon (either the native oxide, a slightly thicker solution-grown oxide or a relatively thicker thermally-grown oxide) or other oxide/hydroxide-bearing materials. The oxide provides the functionality (as hydroxyl and/or silanol groups) to anchor a crosslinked siloxane network. Said network, in turn, serves as the base for a stable, ordered, thin (<30 Å) organic monolayer film. The film may be created by immersing a substrate with a hydroxyl-bearing surface into a dilute organic solution of a long-chain alkyltrichlorosilane (R- (CH₂)ₙ—SiCl₃). The hydrophilic oxide and its physisorbed water layer promote the hydrolysis of the SiCl₃ group, leading to the formation of the anchoring siloxane network. The use of functionalized alkyltrichlorosilanes (X—(CH₂)ₙ—SiCl₃) permits the creation of new, uniformly functionalized surfaces, as illustrated by the following scheme, in which X represents a broad array of functional groups.

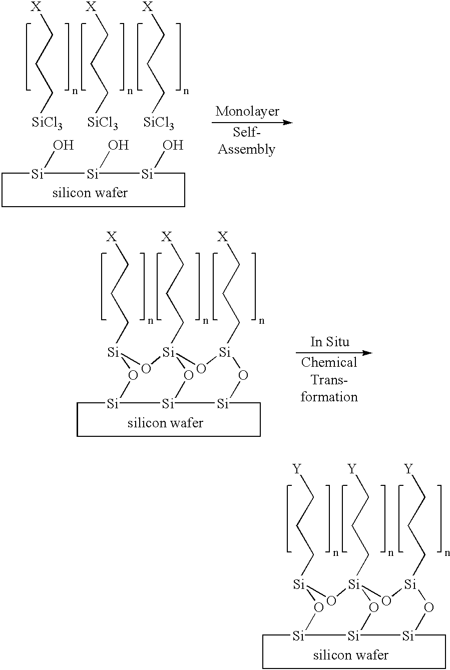

Other strategies for SAM functionalization include the formation of derivatized surfaces made by in situ transformations (e.g., Y =sulfonate, carboxylate, alcohol), whereby a variety of functionalities have been installed on siloxane-anchored SAM surfaces, both by direct deposition and by subsequent in situ transformation. The creation of functionalized SAMs for uniformly oriented peptide attachment is illustrated in Y. W. Lee, J. Reed-Mundell, J. E. Zull and C. N. Sukenik, “Electrophilic Siloxane-Based Self-Assembled Monolayers for Thiol-Mediated Anchoring of Peptides and Proteins”, Langmuir, 1993, 9, 3009-3014. An important recent review by Chechik, Crooks and Stirling provides an excellent summary of much of the chemistry that has been implemented within self-assembled monolayer films (V. Chechik, R M. Crooks and C. J. M. Stirling, “Reactions and Reactivity in Self-Assembled Monolayers”, Adv. Mater. 2000, 12, 1161-1171).

It is important to note that there are a number of other well-documented approaches to SAM formation. Most importantly, Nuzzo, Allara and Whitesides (e.g. E. B. Troughton, C. D. Bain, G. M. Whitesides, R. G. Nuzzo, D. L. Allara and M. D. Porter, “Monolayer Films Prepared by the Spontaneous Self-Assembly of Symmetrical and Unsymmetrical Dialkyl Sulfides from Solution onto Gold Substrates—Structure, Properties and Reactivity of Constituent Functional Groups”, Langmuir, 1988, 4, 365-385 and C. D. Bain, E. B. Troughton, Y. T. Tao, J. Evall, G. M. Whitesides and R. G. Nuzzo, “Formation of Monolayer Films by the Spontaneous Assembly of Organic Thiols from Solution onto Gold”, J. Am. Chem. Soc. 1989, 111, 321-335) pioneered the attachment of a wide array of sulfur compounds (most prominently, thiols and disulfides) to coinage metal surfaces (most prominently, gold). The thiol-on-gold system is not quite as robust as the siloxane-anchored SAMs, but it has proven to be convenient, controllable and informative. It also allows for the direct incorporation of a wide range of surface functionalities (A. Ulman, “Formation and Structure of Self-Assembled Monolayers”, Chem. Rev., 1996, 96, 1533-1554).

Monolayer-modified surfaces have been widely used together with conventional organic chemical techniques for anchoring a wide range of biological materials and functional molecules. There is also a significant body of work wherein an appropriately primed SAM surface provides a template for the attachment of inorganic/ceramic materials (e.g. M. R. DeGuire, T. P. Niesen, S. Supothina, J. Wolff, J. Bill, C. N. Sukenik, F. Aldinger, A. H. Heuer and M. Ruhle, “Synthesis of Oxide and Non-oxide Inorganic Materials at Organic Surfaces”, Zeitschrift für Metallkunde, 1998, 89[11], 758-766; Y. Wang, S. Supothina, M. R. DeGuire, A. H. Heuer, R. Collins and C. N. Sukenik, “Deposition of Compact Hydrous Aluminum Sulfate Thin Films on Titania Particles Coated with Organic Self-Assembled Monolayers”, Chem. Mater., 1998, 10, 2135-2144; and; M. R. DeGuire, H. Shin, R. J. Collins, M. Agarwal, C. N. Sukenik and A. H. Heuer, “Deposition of Oxide Thin Films on Silicon using Organic Self-Assembled Monolayers”, Integrated Optics and Microstructures III, M. Tabib-Azar ed., Proc. SPIE, 1996, 2689, 88-99 and references therein). The novelty of these processes is clear from the ability to use photopatterned sulfonate-SAM surfaces to produce patterned titania films and in the ability to anneal such oxide films (up to 800° C.) with no evidence for delamination of the oxide even when the SAM is burnt away.

SAM functionalization as a route to radionuclide attachment must be contrasted to other current approaches for creating radioactive solids. A drawback of these approaches is that the activation methods used (neutron activation, or activation using high-energy charged-particle beams from a particle accelerator) have limitations of their own. Neutrons are highly penetrating and therefore induce radioactivity throughout a sample, not only on the surface. Particularly in the case of metals, neutron activation produces a mixture of radioactive products because the neutron capture reaction can occur to various degrees with a great many isotopes of most metals. It is a more selective technique when used to activate selected metals added to an otherwise mainly organic solid. Charged particle (proton, deuteron, alpha) beams are easily stopped by matter, and therefore activate only a surface layer. The thickness of the layer, and to some extent, the selectivity of the activation, can be adjusted by adjusting the energy of the impinging particles. The disadvantages of such a technique are that it can cause damage to non-metallic materials because of the high energy deposition process and that the radiation properties one obtains are dependent on the identity of the material and cannot be selected independently. It is also difficult to activate internal or irregular surfaces of a solid.

It is therefore an object of this invention to provide devices, in particular biomedical devices, which include radionuclides in their surface.

Another object is to provide such a device in which the radionuclide is included in a coating that reaches all surfaces of the device.

A further object is to provide such a device in which the radionuclide is included in a coating that holds it sufficiently strongly that no covering layer is required to prevent loss of radionuclide.

A still further object is to provide such a device in which the coating layer is a self-assembled layer.

A still further object is to provide such devices comprising a siloxane-anchored SAM by means of a radioactive nuclide.

It is a still further object to provide devices which have a self-assembled surface, e.g. a SAM surface, that has been functionalized so as to incorporate a radioactive nuclide.

It is a still further object to provide biomedical devices, particularly temporary and/or permanent implants, that include a labeled radioactive surface.

It is a still further object to provide a process for making devices, particularly biomedical devices, that include radionuclides in their surface.

It is a still further object to provide a method for causing siloxane-anchored SAMs to incorporate radionuclides therein.

Other objects and advantages of the invention will appear as the description proceeds.

### SUMMARY OF THE INVENTION

According to the invention, devices of any kind are provided with a surface layer which includes radioactive nuclides. The surface layer is a self-assembled layer, which is preferably chemically anchored. More particularly, the surface layer may be comprised of a siloxane-anchored SAM comprised of a siloxane-anchored SAM or a SAM anchored by other functional groups including, but not limited to, thiols, amines, and phosphonates. The devices preferably comprise a substrate, made of a metal, e.g. stainless steel or silicon, or an alloy, e.g Nitinol, or a non-metallic substance, e.g. quartz, or a polymer.

Incorporation of a radiolabeled molecule into the surface layer of a solid can be done by forming the solid using a radioactive material as in the production of a variety of radioactive sealed sources. Another, preferred method consists of using activation methods to induce radioactivity in a solid after its final formation. This approach avoids the problems of forming radioactive materials, especially the problem which occurs in molten blending, casting and machining radioactive solids, and still provides a final radioactive solid.

According to the invention, radionuclides are incorporated in a self-assembled layer. Self-assembled layers and methods for their creation are known in the art. Apart from the publications hereinbefore cited describing the production of SAMs, other publications describe the production of self-assembled layers other than SAMs. Solution-based coating procedures are included in the references above. Gas-phase procedures that could also be used in this invention, are described in, for example, “Cheng, L; Bocarsly, A. B.; Bernasek, S. L. Ramanarayanan, T. A., Langmuir 1994, 10, 4542”, and in, for example, “Monolayers on Disordered Substrates—Self-Assembly of Alkyltrichlorosilanes on Surface-Modified Polyethylene and Poly(Dimethylsiloxane)”, Ferguson G S, Chaudhury M K, Biebuyck H A, Whitesides G M, Macromolecules, 1993, 26, 5860-5875” The self-assembled layer is preferably, but not necessarily, a SAM. The self-assembled layers reach all surfaces of the device; they allow for controlled incorporation of the radioisotope in a way that also permits the inclusion in the device of other treatment modalities including drugs or anti-thrombotic or anti-fouling molecular units; they retain the radioisotope sufficiently strongly so that no additional covering layer is needed to prevent loss of the isotope; and give access to the convenient incorporation of radioisotopes such as I-131, F-18 and C-11 that have short half-lives and appropriate beta emission energies and can still be conveniently installed on-site (e.g. in a hospital) with no special equipment. These features are highly desirable to obtain the results that the invention intends to obtain.

In a preferred form of the invention, radionuclides are incorporated in a chemically functionalized self-assembled layer, preferably a SAM, which is attached at the surface of a device to which radioactivity is to be imparted. Chemically radiolabeling a specifically functionalized surface, as in a SAM which is attached by a self-assembly process to any surface of a solid, with no limitations on shape or line-of-sight access, brings a degree of control and precision to the radiolabeling process that cannot be achieved by other means. Radioactivity incorporated by a reaction with a functionalized SAM is truly on the surface of the material, not embedded in it and simply near the surface, as it is when other activation techniques are used. Short-range radiation (low energy beta and alpha emissions) can therefore be emitted in directions away from the surface and deposit its entire energy into the surrounding medium. In order to produce a labeled radioactive surface, it is necessary only to functionalize the surface in such a way that a radioactive nuclide in suitable chemical form can be reacted with it in reasonable yield. There are a wide variety of radionuclides available. and the choice is dictated by the physical properties of the nuclide and its radiation.

The invention also comprises a process for making devices having a substrate and a surface layer which includes radioactive nuclides. The process comprises forming the substrate and applying to it a radioactive surface layer. In one form of the process, the surface layer is made of radioactive material. In another form of the process, the surface layer is made of non-radioactive material and is then labeled with a radionuclide. In a preferred embodiment of the process, the surface layer is a chemically functionalized self-assembled layer, more preferably a SAM, incorporating radionuclides. The substrate can be made e.g. of silicon, quartz, Nitinol, stainless steel, cobalt chrome or a polymer. In an embodiment of the invention, the SAM is siloxane-anchored or anchored by thiols, amines or phosphonates.

In an embodiment of the invention, the self-assembled layer is generated by a solution-based process In another embodiment it is generated by gas-based process. As has been said, these processes are described in various publications and are well known to persons skilled in this particular art, so that they need not be described here.

In another embodiment of the process, a solution-based self-assembly of the coating layer is combined with in situ covalent attachment of radionuclides.

### DETAILED DESCRIPTION

A particular, though by no means exclusive, embodiment of the invention consists of a radiolabeled temporary or permanent therapeutic implant, for example a stent for use in angioplasty. For such an embodiment to be fully satisfactory, the radiolabel must emit radiation that will irradiate a desired body surface surrounding the implant—in the case of a stent, to which reference will be made hereinafter, the desired inner wall of the vessel in which the stent is inserted—while minimizing radiation of the surrounding tissue. It is also desirable to have the flexibility to deliver the radiation dose with an intensity and over a period of time that can be readily and precisely varied. Radiation treatment over a short time using sealed radiation sources or external radiation (brachytherapy) of arteries is a method that has been shown to reduce the rate of restenosis (where the occlusion returns) after the artery has been opened. A purpose of this embodiment of the invention is to place a quantity and type of radioactivity on the stent, which will fall into the range that has been shown to be effective in preventing the return of the blockage. It is also preferred to use a labeling procedure that is easily accomplished either on-site in the hospital setting where the angioplasty is performed, or by a commercial external radiopharmacy. The stent is then placed in the artery and the surface of the stent automatically delivers the radiation to precisely the site that must be irradiated to prevent restenosis.

Using SAM-modified surfaces as carriers of this radioactivity has a number of important advantages beyond the uniformity, chemical versatility and durability of the covalently anchored SAM layer. Given the diversity of materials onto which SAMs can be installed, it should be kept in mind that a radiolabeling chemistry that is suitable for a particular set of materials (like the preferred stent-forming metals mentioned hereinafter) can be readily transferred to other materials (e.g., polymers). Further, the very thin (15-30 Å) and compliant nature of the SAM coating guarantees that it will not significantly change the overall geometry of the device to which it is applied and will be flexible enough to follow changes in the size and shape of the device. In the case of a stent, it has been demonstrated that subtle features of the physical structure thereof are important in controlling surrounding cellular processes. One of the important emerging materials for stent fabrication is the shape-memory alloy Nitinol (a titanium-nickel alloy). Since changes in shape with subtle changes in temperature are crucial to the successful operation of such a stent, the aforesaid features of SAMs are important and distinguish SAM-based carriers from other (thicker, physisorbed) polymer-based coatings.

It is not desirable to irradiate tissues that are at a considerable distance from the site of the stent in the artery. Therefore, a short-range radiation is desired. This includes alpha and beta emissions. Gamma emissions from the surface have only a small effect locally, because they are not well absorbed by tissue, and spread the radiation dose over a large volume of the body. So, for therapeutic purposes, the effect of gamma radiation from a labeled stent is negligible. Some gamma radiation could, however, be a desirable feature, as it would provide a means of locating the stent and non-invasively measuring the radioactivity thereof as a function of time by using external nuclear medicine imaging techniques.

There are several nuclides available that could have the desired properties. An essential consideration is whether any of the available nuclides could be expected to produce the necessary local radiation dose using a quantity of nuclide that could at least theoretically be bound to the surface of a SAM. In the case of stents, a dose in the range of 10-20 Gy is desirable. Such a dose has mostly been obtained using materials impregnated with phosphorus-32. When P-32 is used, a source with a strength of 400 KBq is sufficient for therapy. P-32 is a beta-emitter. Therefore, when it is uniformly distributed in a solid source, a significant portion of the radiation dose is absorbed by the material of the source. The radiation dose from P-32 is distributed in a radius equal to the range of its emitted beta particle. At 700 KeV, the range of P-32's beta is about half a centimeter. This fact also implies that a relatively large portion of the dose from P-32 is wasted. The half-life of P-32 is 14.3 days, meaning that the majority of the dose is deposited in a time of about one month. There is literature which indicates that short exposure times, and therefore short half-lives, are desirable. However, in order to determine the possibility of using nuclides other than P-32, relative radiation dose depositions for other nuclides are calculated below relative to the dose deposited by P-32.

Taking into account the various emitted beta particles, their abundance in the total emissions, and neglecting emitted gamma rays, the quantity of each nuclide that would be needed to locally deposit the same energy in the tissue over its lifetime, was calculated. All of the candidate nuclides had beta energies that were less than that of P-32. Therefore, all would be expected to be more effective than P-32 at a given energy deposition. The exact degree of difference that one could expect is not certain because it is not known exactly at what position in the tissue the radiation dose has its effect. Therefore, although it is possible to calculate detailed positional radiation doses, it is believed that total energy deposition is the best comparison value to be used to determine if a particular nuclide could be useful in the application under consideration. A table summarizing the relevant features of various nuclides follows. The table shows the half-lives, the total energy deposition per decay event, and the number of MBq needed of each nuclide (nominal) to theoretically perform effective therapy via a labeled stent. The quantities needed, for the reasons mentioned above, are almost certainly overestimates, except in the case of P-32.

| | | | | Needed |
|---|---|---|---|---|
| Nuclide | t ½ | | MeV Total | (MBq) |
| P-32 | 14.29 | days | 0.7 | 0.37 |
| I-123 | 13.13 | hours | 0.0265 | 9.7848 |
| I-125 | 16.14 | days | 0.0171 | 15.1629 |
| I-13 | 18.04 | days | 0.1814 | 1.4278 |
| H-3 | 12.28 | years | 0.0057 | 45.5585 |
| C-14 | 5730 | years | 0.0495 | 5.2355 |
| S-35 | 87.44 | days | 0.0488 | 5.3041 |
| F-18 | 109.8 | minutes | 0.2416 | 1.0718 |
| C-11 | 20.4 | minutes | 0.3847 | 0.6732 |
| Br-83 | 2.4 | hours | 0.3201 | 0.8091 |
| Br-82 | 35.3 | hours | 0.1361 | 1.9031 |
| Sr-89 | 50.55 | days | 0.5829 | 0.4443 |
| Cu-67 | 61.88 | days | 0.1412 | 1.8342 |
| Cu-64 | 12.7 | hours | 0.1223 | 2.1182 |
| C1-36 | 301000 | years | 0.2475 | 1.0464 |

Several points in the table are important for choosing the most appropriate target nuclide. The best candidates should be very readily available. Iodine-123, e.g., is much less readily available than I-125 or I-131, making it less preferred if all else is equal. Also, those nuclides with a high total MeV deposited are desirable because they will require fewer atoms to be installed within a SAM in order to be effective. This, and the half-life, are the factors that will influence the amount of radioactivity needed for success. The energy of the emitted beta particles (all of the candidates are beta emitters of one kind or another) ideally would give a range of the particle of about 1 mm, which ideally calls for a particle energy of 0.2-0.5 MeV. The last criterion for the choice of nuclide is the half-life. The nuclide should have a half-life such that it deposits the bulk of its radiation dose in a relatively short time, a few hours to a few weeks. Sealed-source brachytherapy is generally applied over a course of minutes, while implanted therapy has most commonly used P-32, which applies the dose over about a month. Half-lives that are much longer will provide a continuing source of radiation, which may represent a hazard and has not been shown to be beneficial. Clinical experience seems to indicate that the benefit of the radiation is achieved in a short time, which implies that it may be most important to cause some specific local cellular change which then can preclude the restenosis response. A short half-life nuclide would therefore apply the necessary radiation dose rapidly during the initial post-implant period, and then would decay away to negligible radiation levels. This criterion eliminates, for example, Cl-36 from serious consideration.

Given the above criteria, strong candidates from the above list are I-131, F-18, C-11, Br-83, Br-82 and Cu-64. The most promising are the first three. I-131 is a nuclide in daily use in most hospitals as a thyroid therapy agent; F-18 and C-11 are routinely used in facilities that perform Positron Tomography. While these last two require a cyclotron for production of the radionuclide, this problem has been solved in most U.S. cities and in many other developed countries by a combination of widely placed cyclotrons and an effective distribution network. In practice, its half-life allows F-18 labeling to be achieved in advance of use by a few hours, and this has been shown to be widely sufficient. While C-11 is produced by the same facilities that produce F-18, its disadvantage is its 20 minute half-life. This demands that production and use be coordinated to within 30 minutes and would limit the generality of this approach. Nevertheless, while logistically more complex, C-11 remains attractive, at least conceptually, because of its rich chemistry for incorporation and its essentially ideal half-life for therapeutic purposes. Finally, I-131 is particularly promising because of its universal availability and convenient, if somewhat longer than ideal, half-life. In extended use, a kit could be provided which would essentially consist of a solid device, e.g. a stent bearing a functionalizable thin film, such as a SAM coating, and the proper amount of oxidant, solvent and other reagents for derivatizing said film with a radionuclide or a radionuclide-containing material. A nuclear pharmacist with standard training in preparing therapeutic and diagnostic nuclear medicine doses, would add the locally obtained radioiodine and the reagents to label the stent and perform the minimal processing and quality control procedures that would be necessary before delivering the labeled stent to the surgeon. In this way, radioactive material is not shipped with the stent, but only used in channels that already exist within the health care system. Alternatively, a kit could be provided including a solid device that is not coated with a functionalizable thin film but rather with means for applying such a film to it, and, optionally, means for derivatizing said film with a radionuclide or a radionuclide-containing material. Such kits, their use and the creation of devices, as claimed herein, are also embodiments of the invention.

Correspondingly, the invention includes a process for the preparation of a device which comprises a surface layer that has incorporated therein at least one radioactive nucleide, which process comprises providing a kit which includes a solid device bearing a functionalizable thin film and the reagents for derivatizing said film with a radionuclide or a radionuclide-containing material, and carrying out the derivatizing when the device is to be used. Also, the invention includes a process for the preparation of a device which comprises a surface layer that has incorporated therein at least one radioactive nucleide, which comprises providing a kit which includes a solid device that is not coated with a functionalizable thin film but rather with means for applying such a film to it, and, optionally, means for derivatizing said film with a radionuclide or a radionuclide-containing material, and carrying out the application of said film and the derivatization thereof when the device is to be used.

The invention further includes a method of applying radioactive radiation to an organ or vascular structure of the human body, which comprises providing a solid device according to the invention, which comprises a surface layer that has incorporated therein at least one radioactive nucleide, and inserting said device into said organ or vascular structure, particularly as a temporary or permanent implant.

The invention further includes such a method, comprising providing a solid device at or near the time and location at which radioactive radiation is to be applied to said organ or vascular structure, and then applying to said device a surface layer that has incorporated therein at least one radioactive nucleide, so as to generate a radioactive device according to the invention, and inserting said radioactive device into said organ or vascular structure, particularly as a temporary or permanent implant.

The other consideration for stent labeling is whether the necessary quantities of radioactive material can be attached to a stent. Iodine-131 would require the largest number of atoms to be attached to the stent. With the iodination chemistry discussed below, it is theoretically possible to attach one iodine to each of the SAM-forming molecules on the surface of a stent. The required quantity of Iodine-131 represents 0.046 nmole, or 2.8E13 atoms. A SAM has a packing density such that a molecule on the surface occupies about 25 square Å, or a square that is 5 Å on a side. For a stent of 7 mm square surface dimension, this is about 2E14 molecules on the surface. Therefore, a loading on the order of ten percent of the molecules should put a sufficient number of iodine atoms onto the stent surface. If one uses carbon-11 or fluorine-18, the same energy deposition overall, and certainly in the first few hours, will be reached with an even lower percentage of loading.

There are standard approaches to radiolabeling with the aforesaid isotopes. A vast number of biomolecules have been labeled with iodine by routine methods. Radioiodide is added to the substrate to be labeled in the presence of an oxidant. The oxidant normally produces iodine, or in any event, an electrophilic iodine species, which is capable of electrophilic attachment to suitable organic sites (generally aromatic rings). In proteins, tyrosine and histidine are the most commonly labeled residues. For this application, the technique needs to be kept simple. Lead tetraacetate and chloramine-T, two commonly used oxidants, are suitable reagents for the reaction. Other oxidizing techniques may be considered. It may also be possible to bind more than one iodine to each aromatic ring on the surface layer. This could lead to a more effective labeled layer, but could also have effects on the structure of the layer.

Labelings with fluorine-18 or carbon-11 are also based on well-established techniques. Fluoride can be reacted directly with an electrophilic monolayer surface, or a nucleophilic SAM terminating in an amine, alcohol, phenol, or carboxylic acid could be reacted with labeled fluoroethyl tosylate in a standard fluorination technique. Likewise, methylation of a variety of nucleophilic functional groups, including those just mentioned, is commonly performed using carbon-11 labeled methyl iodide. Because of the high specific activities of these nuclides, very high radioactivity loads can be placed on the surfaces.

In designing the specific chemistry for creating the monolayer films and for their in situ modification, the first consideration is the nature of the substrates to which the SAMs will be attached. Silicon wafers and quartz (as model substrates) and Nitinol (as a currently important biomaterial) all have surface layers of either silicon oxide or titanium oxide. While the specific cleaning and priming procedures are adjusted to assure effective SAM attachment, all of these substrates are well suited for standard siloxane-anchored SAMs based on functionalized alkyltrichlorosilanes. On the other hand, stainless steel and cobalt chrome have native oxide layers which have proven to be less useful for SAM attachment. A number of different approaches can be employed for SAM construction on their surface. In one approach, the native oxides of these metals are enhanced by first applying an overlayer of silicon oxide. This approach has also been proven effective in allowing SAM attachment to otherwise inert polymer surfaces. However, such oxide enhancement (using a vapor phase treatment with SiX₄) and the actual SAM attachment chemistry (using functionalized RSiX₃) cannot involve systems where X=Cl, due to corrosion considerations. Alkoxysilanes, with hydrolysis conditions that optimize the formation of a controlled uniform surface layer, may be used. This affords the advantage of avoiding chloride ions when working on corrosion-prone substrates like stainless steel. Another approach to coating stainless steel and related substrates is based on removal of the surface oxide and attaching the SAM to the bare metal. In this case, the use of thiol and/or amine-anchored SAMs is required.

The interplay of the above-defined anchoring chemistry with the chemistry required to install the specific radionuclides will now be considered. Radioactive iodine is best attached specifically to aromatic rings, because the resulting aryl iodides are so much more hydrolytically stable than alkyl iodides. The preferred route is standard for radioiodination, by electrophilic iodination using radioiodide and a suitable oxidant to convert iodide to I₂. It is also desirable to use an aryl functionality on the SAM that is suitably activated towards electrophilic substitution. Iodine can be incorporated onto monolayers on stainless steel, but with less harsh conditions than applied to monolayers on silicon/glass. Harsh conditions include the use of oxidants like lead tetra-acetate in solvents like acetic acid or acetic acid/water mixtures, at temperatures as high as 80° C., to transform the iodide ions into the required electrophilic iodination reagent. Less harsh conditions, which have proven advantageous in avoiding monolayer damage, include the use of oxidants like copper salts and air or peroxides or chloramine T, at temperatures as low as 25° C.

A problem to be considered for radio-fluorine installation is that, when a fluoride is used on a siloxane-anchored SAM, the fluoride may attack the anchoring siloxanes. However, the monolayer affords some protection to the siloxane network, such that it can survive short exposure to fluoride. Given the stability of primary alkyl fluorides and the ease with which they can be formed by reaction with simple alkyl halides or sulfonates, they remain attractive for the purposes of this invention. Monolayers with sulfonate leaving groups lead to the direct incorporation of fluoride provided by reagents such as tetrabutyl ammonium fluoride. Solvents for this process include acetonitrile, dimethylformamide and others.

As to thiol or amine-anchored films, the SAM forming molecules should containing a thiol or amine at one end (for anchoring) and an electrophilic terminal group for trapping fluoride. In order to coexist with the anchoring group, the electrophile should be protected until the SAM is established on the metal surface. In general, it is also possible to first incorporate the fluoride into a suitable electrophilic derivative and then attach it to a nucleophilic surface compatible with either siloxane or thiol/amine-anchored SAMs.

The attachment of siloxane-anchored SAMs to the silanols on the surface of silicon wafers, glass, and quartz, is well precedented. The present inventors have also demonstrated the secure attachment of such films to the surface of titanium oxides both as rutile particles and on the native oxide of titanium metal and a number of its alloys. This chemistry has been extended to the oxide surface of the TiNi alloy. Alkyl phosphonates and phosphates have also proved to be effective to anchor functionalized SAMs onto TiNi and other oxide-bearing surfaces.

The anchoring of the monolayer on stainless steel or related substrates, like cobalt chrome, is preferably enhanced by one of the two following procedures. The first procedure consists of enhancing the surface oxide of said substrates by vapor deposition of a silanol layer by exposure to silanes other than SiCl₄, which could cause pitting corrosion on the metal substrates, e.g. to vapors of tetra-alkoxy and tetra-acetoxy silane. Alternatively, solution-based procedures for creating an SiO₂ layer can be used. The second procedure consists of the reductive removal from the substrate surface of the oxides to provide a surface that adsorbs SAMs using either amine or thiol headgroups. These inventors have successfully established such SAMs. The C—H stretching frequencies (measured by reflection IR spectroscopy on reduced stainless steel substrates) for such SAMs, as a function of head group and length of the alkyl chain of the SAM forming monomer, are tabulated below. The peak intensities are, as expected, proportional to chain length. There is a trend towards lower frequency CH₂ adsorptions, suggestive of more crystalline monolayer packing, as a function of increasing chain length.

### Infrared Peak Positions for n-alkanethiol and n-alkylamine Monolayers on 316 Stainless Steel

CH₂

| | Vₛ | |
|---|---|---|
| Monolayers | (cm⁻¹) | Vₐ (cm⁻) |
| C₁₈SH | 2851 | 2921 |
| C₁₈NH₂ | 2851 | 2922 |
| C₁₆SH | 2852 | 2922 |
| C₁₆NH₂ | 2852 | 2922 |
| C₁₂SH | 2853 | 2923 |
| C₁₂NH₂ | 2853 | 2924 |
| C₁₀SH | 2854 | 2924 |
| C₁₀NH₂ | 2855 | 2925 |

These results show that it is possible to form satisfactory monolayer films using either thiol or amine anchors. A range of chemical and electrochemical procedures are also suitable for priming the surfaces.

A general methodology for surface iodination can be based on the electrophilic iodination of surface-anchored phenyl ethers. The synthesis of the phenyl ether SAM-forming compounds is based on the reaction of phenol with an alkyl bromide or mesylate. Commercially available alcohols and bromides allow easy access to such molecules with different length alkyl chain tethers ranging from 3 to 16 carbons.

The synthesis of an alkyl phenyl ether with terminal olefin is schematically indicated as follows:

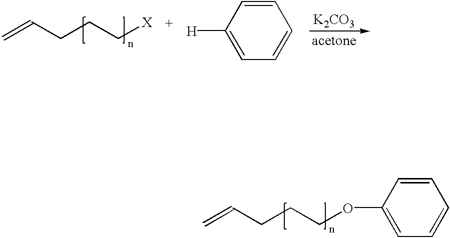

The terminal olefin provides a convenient handle for the installation of surface anchoring functionality (most conveniently, thiols phosphonates_or silanes) and the specific embodiment of this system on a silanol bearing surface is shown in the scheme below.

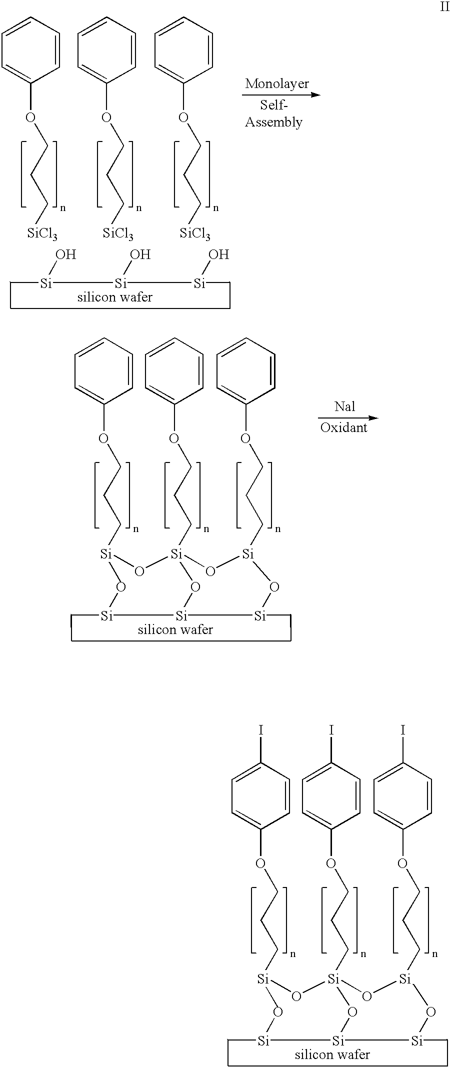

The C-11 phenyl ether has been successfully made and its trichlorosilanes have been anchored to silicon wafers and to quartz plates. The wetting properties of these films have been assessed and they have been studied by ellipsometry and FTIR on the silicon wafers and by UV spectroscopy on quartz plates. The iodination chemistry using NaI and Pb(OAc)₄ in mixed acetic acid/water solvent has been demonstrated, as determined by the iodine signal that appears in the XPS of the films and by the change in the UV spectrum from that of the phenyl ether to that of the para-iodo-phenyl ether (comparable to that of authentic). Using the same reagents (though with a much lower concentration of iodine), radio-iodine has been incorporated into the films in such a fashion that it does not wash out. Pb(OAc)₄ and chloramine T are examples of oxidants, but other oxidants can be used. The rate of the iodination chemistry has been found to be solvent and temperature-dependent.

The ability to count the incorporated radioactivity (for a fixed size silicon wafer) is a powerful way to quantify the reaction yield and to carefully optimize the reaction conditions.

The same iodination chemistry can be applied to siloxane-anchored films on Nitinol and on stainless steel. The same alkyl phenyl ether with the terminal olefin can be used in the trialkoxy-silane-based, siloxane-anchored route to stainless steel labeling. It also can be used to synthesize the terminal thiol SAM forming moiety.

An alternative to the direct monolayer iodinations described above can be found in the possible application of the Bolton-Hunter reagent. This reagent is often used for iodination of proteins with exposed amino groups. The reagent is the N-hydroxysuccinimide ester of 3-(4-hydroxyphenyl)propionate, and it would react with amine terminated monolayer surfaces much as it has been shown to react with lysines in proteins. The aromatic ring is labeled via oxidative iodination conditions. The very activated positions ortho to the hydroxyl are labeled. Two iodines can even be placed on one molecule to get the 3,5-di-iodo material in which both iodines are radioactive. The reagent can be prepared as needed and is also available commercially in the radiolabeled form.

As indicated earlier, the constraints on fluoride incorporation are quite different and in some ways complementary. The stability of alkyl fluorides permits simple nucleophilic substitution chemistry. Thus, alkyl bromide or iodide SAMs are excellent substrates for simple SN2 chemistry using a fluoride nucleophile. The problem of the fluoride ion attacking a siloxane anchoring network may be minimized by a well-packed SAM using long chain alkyl tethers and the use of fluoride (with no carrier added—NCA) as a limiting reagent. Also, more aggressively electrophilic surfaces bearing halo-acetyl groups may further tip the balance in favor of fluoride incorporation without SAM delamination.

An attractive approach to F-18 installation into siloxane-anchored films is through the intermediacy of fluoroethyl tosylate. The ditosylate is reacted with fluorine-18 fluoride, which in turn is prepared in the cyclotron target from the O-18(p,n)F-18 nuclear reaction and dried by azeotropic distillation using acetonitrile. The reaction is done in acetonitrile solution (freshly distilled from CaH₂). The resulting fluoroethyl tosylate is purified by HPLC to remove the precursor, and reacted with the monolayer after evaporation of the HPLC solvent. The entire procedure to the point of reacting with the monolayer requires about 40 minutes. Given that a variety of nucleophilic SAMs that react with primary tosylates are available, it takes no more than one hour to produce a labeled monolayer. In this way, a siloxane-anchored SAM can be impregnated with F-18 without ever being subjected to treatment with fluoride ion. This is illustrated in the following figure. Conceptually, the idea of pre-labeling an auxiliary material and then reacting it with a suitable SAM is analogous to the described use of the Bolton-Hunter reagent, above.

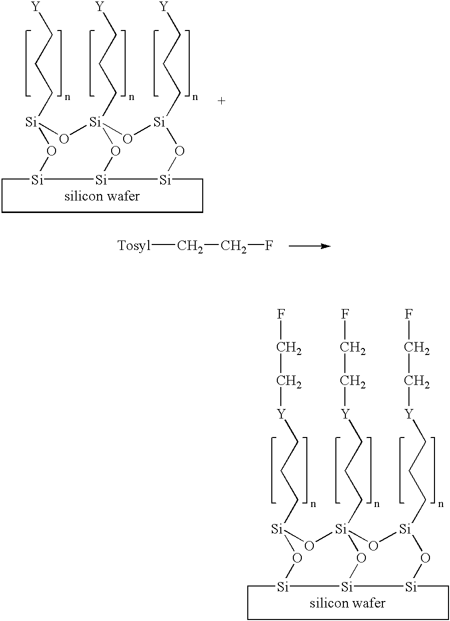

This chemistry, as schematized above, could also be applied to thiol-anchored SAMs using commercially available [symbol] ,ω hydroxythiols or the readily prepared hydroxylamine analogues, wherein the exposed hydroxyl groups on the monolayer reacts with the tosylate. Given that the thiol-anchored SAMs have no problem with fluoride ion, an equally effective approach is to attack an electrophilic SAM surface with fluoride. Implementing this by using □,ω functionalized thiols with electrophilic groups may encounter stability problems with the SAM forming materials (having an electrophile in the same molecule as a thiol). It is therefore preferable to install the surface electrophile using in situ chemistry that can be implemented after monolayer formation but will still result in a stable surface. The inventors have found that chloroacetyl units are excellent electrophiles and yet have good stability under ambient conditions; and have determined (with siloxane-anchored SAMs) that OH-terminated SAMs can be elaborated by the in situ reaction of the hydroxyl-bearing surfaces with alpha-chloro-acetyl chloride to produce the chloroacetate electrophiles at the monolayer surface. This same chemistry is readily applicable to the OH-bearing thiol-anchored SAMs described above. The chloroacetyl groups thus installed is expected to react instantly with fluoride ion and provide for simple nucleophilic labeling with F-18. The very short exposures to small amounts of chloride that are required by this chemistry is expected not to cause undue corrosion when applied to stainless steel surfaces. A similar result is achieved by reacting a SAM with exposed hydroxyl groups with triflic anhydride followed by fluoride incorporation.

The self-assembly process can also be used to create mixed monolayer films. Monolayer self-assembly using a solution containing molecules with the same anchoring functionality, but with more than one kind of terminal functional group or with two or more chain lengths, is well known. It can have important ramifications for this invention, both in terms of the design and optimization of systems for radionuclide incorporation and for creating therapeutic tools with functional surfaces that can deliver a number of desirable effects simultaneously. A scheme that will facilitate the discussion of these issues is shown below.

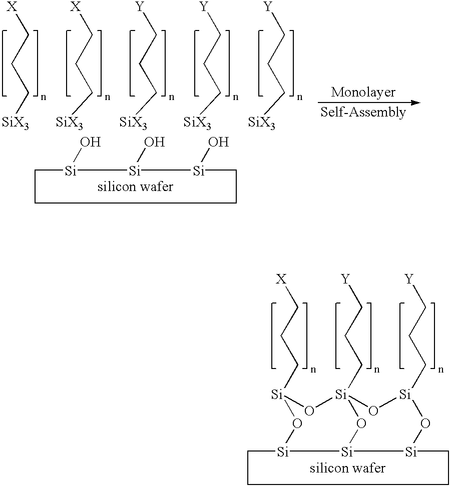

The above scheme uses a siloxane-anchored SAM as an example, though the concept has been amply demonstrated for thiol-based SAMs as well. It depicts a system wherein two different SAM forming monomers (bearing X and Y groups, respectively) are mixed in the homogeneous deposition fluid and the SAM substrate (i.e., the silicon wafer) is immersed into this mixed medium. It correctly represents the known observation that the percentage of each monomer deposited on the surface is not necessarily the same as the percentage of that monomer in solution.

The simple example is that wherein X represents a phenyl ether and Y represents a simple (methyl-terminated) alkyl chain. Alternatively, various functionalities of the Y groups can create environments of different polarities and solvent properties around the X group. Varying the reaction solvent (even as subtly as changing the ratio of HOAc and water) has been shown to significantly change the reaction rate for the iodination chemistry above. Further, short chain Y molecules as spacers are expected to allow the phenyl ethers to be more accessible to the solution chemistry, while using chains longer than the length of the phenyl ether molecule itself might fold these chains over the targeted chromophore and retard its reaction. UV spectroscopy allows us to measure the actual ratio of phenyl ether to alkyl chain in the surface film and can also (somewhat crudely) monitor the progress of the reaction. The radioactivity assay is a much more precise measure of the rate and extent of reaction in these various systems.

The above-described dilution experiments should help optimize the radiolabeling process. The inventors have calculated that for the case of radio-iodine, a 10% loading is sufficient to achieve therapeutically effective doses. For F-18 and C-11, this loading could be even lower. Thus it is possible to mix various radioactivity-incorporation sites (at low concentration) with functionality that would prevent thrombosis or limit adventitious protein adsorption (dominating the composition of the surface). The effectiveness of SAMs to control protein and cell adsorption has been demonstrated and such control on a stent surface could influence its effectiveness. With the right composition surface on a stent, restenosis could be additionally inhibited just by the surface. It is well known that some surfaces cause more of a clotting reaction than others, and a major focus in implantable biomaterial design is the control of such surface processes.

Another possibility is to dilute the surface with Y-bearing molecules containing sites that can attach a range of biomolecules including enzymes and/or growth factors. Similarly, dilution may provide a vehicle for localized drug delivery. The chemistry for the attachment of more complex molecules to a SAM is well established (see for example Y. W. Lee, J. Reed-Mundell, J. E. Zull and C. N. Sukenik, “Electrophilic Siloxane-Based Self-Assembled Monolayers for Thiol-Mediated Anchoring of Peptides and Proteins”, Langmuir, 1993, 9, 3009-3014).

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A device, which comprises a surface layer that has incorporated therein at least one radioactive nuclide.

2. A device which comprises a substrate and a self-assembled layer that has incorporated therein at least one radioactive nuclide.

3. A device according to claim 2, wherein the substrate is selected from the group consisting of stainless steel, Nitinol, silicon, quartz, cobalt chrome and polymers.

4. A device according to claim 1, wherein the self-assembled layer is an anchored SAM.

5. A device according to claim 2, wherein the anchored SAM is selected from the group consisting of monolayers or films anchored by siloxane, thiol, amine or phosphonate.

6. A device according to claim 1, comprising a substrate of a metal selected from the group consisting of stainless steel and Nitinol and a self-assembled layer anchored by phosphonate.

7. A device according to claim 1, wherein the surface layer is formed of a radioactive material.

8 A device according to claim 1, wherein the surface layer is formed of a radioactive material that has been activated to induce radioactivity therein after its final formation.

9. A device according to claim 1, which comprises a chemically functionalized SAM incorporating radionuclides attached at the surface of the device.

10 A device comprising a substrate covered on all its surfaces by a self-assembled layer, which layer includes radioactive nuclides, and having no other protective layer or coating over said self-assembled layer.

11. A temporary or permanent therapeutic implant, comprising a substrate and a radioactive self-assembled surface layer.

12. An implant according to claim 11, wherein the self-assembled surface layer is an anchored SAM.

13. A device according to claim 11, which is a stent for use in angioplasty.

14. A device according to claim 11, wherein the surface layer has a thickness of less than 10 nm.

15. A device according to claim 11, wherein the substrate is made of Nitinol.

16. A device which comprises a substrate and a self-assembled layer that has incorporated therein at least one radioactive nuclide, wherein the nuclide is selected from the group consisting of I-131, F-18, C-11, Br-83, Br-82 and Cu-64.

17. Process for making a device according to claim 1, which comprises providing a substrate, forming on the substrate a self-assembled surface layer, and providing said self-assembled layer with a radioactive material.

18. Process according to claim 17, wherein the surface layer is made of radioactive material.

19. Process according to claim 17, wherein the surface layer is made of non-radioactive material and is then labeled with a radionuclide.

20. Process according to claim 17, wherein the self-assembled surface layer is a chemically functionalized SAM incorporating radionuclides.

21. Process according to claim 17, wherein the self-assembled surface layer is labeled with iodine.

22. Process according to claim 21, wherein the surface layer is labeled with iodine by adding radioiodide in the presence of an oxidant.

23. Process according to claim 14, wherein the [substrate] surface layer is labeled with fluorine-18 or carbon-11.

24. Process according to claim 15, wherein the substrate is made of a material selected from the group consisting of silicon, quartz and Nitinol.

25. Process according to claim 20, wherein the SAM is a siloxane-anchored SAMs based on functionalized alkyltrichlorosilanes.

26. Process according to claim 29, wherein the substrate is made of a material selected from the group consisting of stainless steel and cobalt chrome which have native oxide layers.

27. Process according to claim 26, wherein the native oxides are enhanced by applying an overlayer of silicon oxide.

28. Process according to claim 17, wherein the self-assembled surface layer is formed by a solution-based process.

29 Process according to claim 17, wherein the self-assembled surface is provided with a radioactive material by in situ covalent attachment of radionuclides.

30. A kit for the preparation of a device according to claim 1, which comprises a solid device bearing a functionalizable thin film and the reagents for derivatizing said film with a radionuclide or a radionuclide-containing material.

31. A kit for the preparation of a device according to claim 1, which comprises a solid device that is not coated with a functionalizable thin film but rather provides the means for applying such a film to it, and, optionally, means for derivatizing said film with a radionuclide or a radionuclide-containing material.

32. A process for the preparation of a device according to claim 1, which comprises providing a kit which comprises a solid device bearing a functionalizable thin film and the reagents for derivatizing said film with a radionuclide or a radionuclide-containing material, and carrying out the derivatizing when the device is to be used.

33. A process for the preparation of a device according to claim 1, which comprises providing a kit which comprises a solid device that is not coated with a functionalizable thin film but rather provides the means for applying such a film to it, and, optionally, means for derivatizing said film with a radionuclide or a radionuclide-containing material, and carrying out the application of said film and the derivatization thereof when the device is to be used.

34. Method of applying radioactive radiation to an organ or vascular structure of the human body, which comprises providing a device according to claim 1, and inserting said device into said organ or vascular structure.

35. Method of applying radioactive radiation to an organ or vascular structure of the human body, which comprises providing a solid device at or near the time and location at which radioactive radiation is to be applied to said organ or vascular structure, applying to said device a surface layer that has incorporated therein at least one radioactive nucleide, whereby to generate a device according to claim 1, and then inserting said radioactive device into said organ or vascular structure.
